(19) United States
(12) Patent Application Publication
(10) Pub. No.: US 2006/0062735 A1
(43) Pub. Date: Mar. 23, 2006

(54) Combination therapy and means for carrying out said therapy

(76) Inventors: Werner Wessling, Rengsdorf (DE); Thomas Hille, Neuwied (DE)

Correspondence Address: William F Lawrence;Frommer Lawrence & Haug, 745 Fifth Avenue, New York, NY, 10151, US

(21) Appl. No.: 10/520,152

(22) PCT Filed: Jun. 12, 2003
(86) PCT No.: PCT/EP03/06146

§ 371 Date: Jan. 3, 2005

(30) Foreign Application Priority Data
Jul. 5, 2002 (DE) 10230558.7

(51) Int. Cl. A61L 9/04; A61L 15/16

(52) U.S. Cl. 424/45; 424/448

(57) ABSTRACT

Disclosed is a combination therapy comprising permanent administration of a transdernal therapeutic system (TTS) in order to provide a patient with a basic supply of a pharmaceutical agent and initial administration of the same pharmaceutical agent by means of a spray, allowing the lag time observed in a TTS therapy to be reduced. Also disclosed is a set comprising a TTS and a spray for the inventive combination therapy.

The invention relates to the administration of pharmaceuticals. In particular, the invention relates to a combination treatment by means of TTS and simultaneous treatment by means of an aerosol (spray, metered aerosol, pharmaceutical metered aerosol), the action of the medicament commencing without or only with a slight time delay. The combination is particularly advantageous for the treatment of severe or chronic pain, as even in basic therapy “breakthrough pain” can occur which necessitates a rapid administration of pharmaceuticals, in this case analgesics. The combination therapy thus comprises a continuous administration of a transdermal therapeutic system (TTS) for the basic supply of a patient with a pharmaceutical active compound and an initial administration of the same pharmaceutical active compound by means of an aerosol. By this means, the lag-time observed in the case of a therapy with a TTS can be reduced. Also disclosed is a set comprising a TTS and an aerosol for this combination therapy.

A disadvantage faced by the undoubtedly great advantages which the transdermal administration of pharmaceuticals (pharmaceutical active compounds) has is often not only the qualitative and quantitative limitation of pharmaceuticals which can be absorbed by the skin, but also the fact that the absorption by the skin only commences with a great time delay. It is known to the person skilled in the art that the skin is not an absorption organ, but rather has the object of preventing the penetration of foreign bodies, that is even of pharmaceuticals. Since these facts are known to the person skilled in the art, the idea of the “lag-time” was coined. This is understood as meaning the time between the first administration of a transdermally administrable medicament, e.g. of a transdermal therapeutic system (TTS), and the first occurrence of a measurable plasma concentration or the first occurrence of the expected physiological action of the pharmacon. This lag-time is particularly critical if a pharmaceutical is not only to be administered chronically for continuous use, i.e. over a relatively long period of time, but if it is simultaneously also required that its action should commence as immediately as possible after the first administration of the medicament, e.g. in the case of the administration of centrally active painkillers. Admittedly, the disadvantageous lag-time can be circumvented or reduced by additionally administering, in the case of the administration of a TTS for the first time or in the case of breakthrough pain, a medicament having a rapid release of active compound, e.g. an oral solution or an intravenous injection. Such a combined administration, however, is not unproblematical, since an intravenous injection must compulsorily be performed by a physician. The administration of tablets combined with simultaneous administration of TTS is also not helpful, since the oral absorption of opiates also only commences with a delay. An alternative would be the administration of solutions; however, it is disadvantageous here that the dosage is left to the patient, which is problematical in the case of opiates (addictive potential of the opiates).

Therefore, simultaneously to the start of the development of therapy by means of dermal or transdermal administration, ways were sought of treating the breakthrough pain, by treating the TTS with ultrasound or by heat development. These processes have the disadvantage that they are not simple. They have therefore not gained acceptance in practice, since hitherto there is neither an ultrasonically assisted TTS nor a TTS which is equipped with a heating element.

Other possibilities for increasing the absorption rate of pharmaceuticals consist in the removal of the stratum corneum by laser treatment or by repeated sticking on and tearing off of adhesive strips, or “stripping”. Both these methods of treatment admittedly also shorten the lag-time, but in this process it is disadvantageous that not only the desired penetration of the pharmaceutical, but also an undesired penetration of other constituents of the medicament and of microorganisms, such as bacteria, fungal spores etc., into the human body is facilitated. The process furthermore has the disadvantage that the TTS has to be removed in order to “strip” the skin. Among experts it is known, however, that the peeling off of a TTS leads to the loss of the adhesive power, since the uppermost layer of skin, which rests on the adhesive of the TTS, is also additionally removed.

A further way to improve the dermal absorption rate consists in the use of current. This process, known under the term iontophoresis, as is known to the medical person skilled in the art, cannot be used pain-free. The “prickly patch” can likewise not be used pain-free. This form of a dermal medicament is fixed to the body by needles which penetrate the skin. The release of active compound takes place through the needles, which simultaneously serve as fixing aid. It is obvious that the discussion here can no longer be of dermal or transdermal administration in the classical sense of the word, but of subcutaneous injection of a pharmaceutical, with all its known disadvantages (necessity of sterile needles, no protracted release etc.).

An alternative which is of interest at first glance is “phonophoresis” or “sonophoresis”. These are understood as meaning the channeling of pharmaceuticals by the living skin into underlying tissue by means of ultrasound. Beyond a number of different studies in orthopedics and sports medicine, routine therapeutic use is not known.

The object of the invention is therefore the provision of a therapeutic process and a means for carrying out this process, in which a pharmaceutical is to be administered to a patient for continuous use, i.e. over a relatively long period of time, where at the same time the action is to commence as immediately as possible after the first administration. In other words: The lag-time should be minimized. Furthermore, in periods of time which occur during the continuous use and are characterized by an increased pharmaceutical need, an additional administration of a pharmaceutical should be brought about with a minimum lag-time.

The object is achieved according to the invention by a process which comprises the simultaneous administration of a transdermal therapeutic system (TTS) with a first pharmaceutical and of a spray (aerosol) which contains either the same pharmaceutical as the TTS or a second pharmaceutical which is at least suitable for the same indication. Whereas subsequently a TTS can be administered for continuous use over a relatively long period of time (for covering the basic need), the aerosol is employed for a bolus administration once at the start of the therapy and optionally additionally at periods of time which occur during the continuous use and are characterized by an increased pharmaceutical need.

In a particular embodiment of the process, this is used for the treatment of pain. This pain can be chronic and/or acute states of pain.

In the following, the technical terms used are to be explained in greater detail. The term medicament is known to the person skilled in the art. This is understood as meaning substances or mixtures of substances for human or veterinary medicine. They consist of the pharmaceutical active compound(s) and further customary constituents which make this active compound pharmaceutically usable. The pharmaceutical active compounds which can be utilized according to the invention are those which are transdermally administrable, since it can also be expected of these that they are rapidly absorbed via the mucosa of the mouth, the lungs or the nasal mucous membrane. A prerequisite for this is that the active compounds, preferably opioids, are highly active, i.e. that the daily dose is in the milligram range and that their pharmaceutically acceptable salts are readily water-soluble (preferably greater than 10%).

By analgesics within the meaning of the present invention, pharmaceuticals are intended which in therapeutic doses reduce or suppress the sensation of pain. These include, in particular, strongly active analgesics which affect the central nervous system, the “opioids”. This group of pharmaceutical active compounds includes, inter alia, morphine, heroin and further derivatives of morphine, dihydromorphine derivatives, such as hydromorphone, oxycodone, morphinan derivatives such as levorpharnol, buprenorphine, analgesics of the pethidine group, such as pethidine, ketobemidone, methadone and derivatives such as levomethadone, dextromoramite, fentanyl and its derivatives, benzomorphan derivatives such as pentazozine and phenylaminocyclohexynyl derivatives such as tilidine.

It is obvious that the practical use of the present invention is of particular importance for the administration of analgesics, since in the acute state of pain it is unreasonable for the patient to wait to the end of the lag-time until the action of the medicament commences. In such a case, a possible acceptable lag-time is a period of time of up to a few minutes.

The device according to the invention for transdermal therapy comprises a transdermal therapeutic system (TTS) comprising a first active compound having a low skin penetration rate and a spray (aerosol) which contains this first active compound or optionally another active compound which is suitable at least for the same indication, preferably in the form of a readily water-soluble salt. The device is preferably present as a set which contains at least one TTS and at least one aerosol. In a further embodiment, the TTS is constructed in layer form and can contain a layer of a pressure-sensitive contact adhesive, a porous layer or a layer of a hydrogel. As a pharmaceutical active compound having a low skin penetration rate, the device can contain an analgesic.

The aerosol can, without restricting the invention, be a nasal spray or a spray for oral administration. The other active compound optionally used can be a pharmacologically equivalent medicament, in the case of analgesics, for example, another opioid.

The invention is illustrated by the following example. A TTS containing buprenorphine, as described in DE 39 39 376, is administered to a patient in pain. At the same time, the patient receives a spray for nasal or oral administration, which contains a 10% strength aqueous solution of buprenorphine tartrate and a thickener (collidone). The high concentration of the pharmaceutical in the spray makes it possible for the patient, in the case of breakthrough pain, to experience immediate relief in the acute state of pain by means of the administration of one or more metered puffs into the pharynx or into the nose, as a result of rapid absorption of the water-soluble salt.

What is claimed is:

1. A process for the administration of a pharmaceutical active compound to a patient who is dependent on the administration of this active compound over a relatively long period of time, comprising
a) administration of a transdermal therapeutic system (TTS: comprising said active compound at the start of the relatively long period of time, and
b) simultaneous administration of this active compound or of another active compound which is suitable for the same indication, a single time, in the form of an aerosol.

2. The process according to claim 1, characterized in that during the relatively long period of time further transdermal therapeutic systems are administered at regular intervals to maintain the basic supply of the active compound.

3. The process according to claim 1, characterized in that during the relatively long period of time at least one time section occurs with an increased need of the active compound for the patient, characterized in that during this at least one time section a further administration at least a single time of this active compound or of another active compound which is suitable for the same indication takes place in the form of an aerosol as a bolus administration.

4. The process according to claim 1, characterized in that the patient who is dependent on the administration: of an active compound over a relatively long period of time suffers from the symptoms of pain, asthma, diabetes, risk of cardiac infarct, overcoming nicotine addiction or Parkinson's disease.

5. The process according to claim 1, characterized in that the active compound is an analgesic, a broncholytic, an antidiabetic, a vasodilator, an agent for overcoming addiction or an antiparkinson agent.

6. A set comprising a transdermal therapeutic system (TTS) containing a first active compound and an aerosol containing the first active compound or another active compound which is suitable for the same indication.

7. The set according to claim 7, characterized in that the first active compound is selected from the group comprising analgesics, broncholytics, antidiabetics, vasodilators, agents for overcoming addiction and antiparkinson agents.

8. The use of a pharmaceutical active compound for the production of a set consisting of:
a) at least one transdermal therapeutic system (TTS) containing this active compound, and
b) at least one aerosol containing the same active compound or another active compound which is suitable for the same indication,
for the treatment of a patient who is dependent on the administration of this active compound over a relatively long period of time, where the transdermal therapeutic system (TTS) and at the same time the said active compound or the other active compound which is suitable fn:: the same indication are administered in the form of an aerosol at the start of the relatively long period of time.

9. The use according to claim 8, characterized in that the patient who is dependent on the administration of an active compound over a relatively long period of time suffers from the symptoms of pain, asthma, diabetes:;, risk o5 cardiac infarct, overcoming nicotine addiction or Parkinson's disease.

10. The use according to claim 8, characterized in that the active compound is an analgesic, a broncholytic, an antidiabetic, a vasodilator, an agent for overcoming addiction or an antiparkinson agent.
